(19) United States
(12) Patent Application Publication
(10) Pub. No.: US 2006/0195341 A1
(43) Pub. Date: Aug. 31, 2006

(54) Method and system for creating a conveniently accessible medical history

(76) Inventors: Alan Haaksma, Oakville (CA); Dustin Thomas Ide, Mississauga (CA)

Correspondence Address: BINGHAM MCHALE LLP, 2700 MARKET TOWER, 10 WEST MARKET STREET, INDIANAPOLIS, IN, 46204-4900, US

(21) Appl. No.: 10/542,343

(22) PCT Filed: Jan. 15, 2003
(86) PCT No.: PCT/CA03/00031

§ 371 Date: Apr. 17, 2006

(51) Int. Cl. G06F 19/00; A61B 5/00

(52) U.S. Cl. 705/3

(57) ABSTRACT

A method and system for creating a conveniently accessible medical history. An information-input node receives medical information and transmits it through an information-transmission connection to a record-creating node, which is remote from the information-input node. The record-creating node is for receiving the information through the information-transmission connection, for configuring it into a medical history record, and for transmitting the medical history record. There is also a record output node, remote from the record-creating node, for receiving the medical history record. The record may be stored on a portable readable storage medium.

For the purposes of the United States of America, this application is a continuation-in-part of U.S. application Ser. No. 09/925,571 filed Aug. 9, 2001.

### FIELD OF THE INVENTION

This invention relates to the field of personal medical information management, and in particular, to the field of computerized personal medical information management.

### BACKGROUND OF THE INVENTION

A medical emergency can happen to anyone. Unfortunately, there is today a wide variety of potential causes for medical emergencies. For example, as the population ages, heart disease is increasing, and accompanying this rise in heart disease is an overall increase in the incidence of acute heart attack. Statistics show that heart disease is the number one killer of people in the United States. Heart disease and acute heart attack lead to millions of hospital emergency room visits and physician office visits each year. In the emergency room setting, the quality of treatment for acute heart attack is significantly increased if the caregiver has access to relevant medical information such as the patient's most current electrocardiogram, his current medications and his medical history.

Other medical conditions that could lead to emergency situations include allergies, epilepsy, diabetes, and adverse drug reactions. In each case, the effectiveness of treatment would be increased by knowledge of what medications the patient may be taking, the results of tests that the patient may have recently undergone, or the patient's medical history.

Even in non-emergency situations, it is frequently important for a treating physician to know facts from the patient's medical history. While the patient's regular physician will usually have access to most such data, this may not be the case when the patient is being treated by a physician other than his regular physician, such as, for example, a specialist.

In the event that information on a patient's medical background is required, the attending caregiver has the option of attempting to contact the patient's regular doctor, or health maintenance organization (HMO), in order to obtain the necessary information. However, this procedure suffers from some important defects. First, particularly in emergency situations, time is of the essence. Attempts to reach other doctors to obtain relevant information can be time consuming, and the information may come too late to help the patient.

Second, there is no guarantee that the attending caregiver will even know who the patient's regular doctor is, or where to obtain the patient's medical history. After all, the patient may be unconscious, or otherwise unable to communicate.

Third, there are significant concerns relating to patient confidentiality in any system where an attending caregiver simply calls the patient's regular caregiver or HMO to obtain information. Under such a system, any person can contact a doctor or HMO, pretending that there is an emergency in progress and that the medical records of a certain patient are required. To remedy this it might be possible to implement some kind of password system which would require the HMO or regular doctor to be given a password prior to the release of medical information. However, in an emergency situation, the patient may not be able to communicate what his password is and the attending caregiver may not have any other way of knowing what the password is.

Fourth, it is possible that the patient's medical history is not centralized, so that some aspects of the medical history would be stored with one doctor, specialist or organization, and some aspects with others. Thus, in an emergency, the attending caregiver may actually need to phone around to a number of different locations to obtain the required information. This would be unacceptable, particularly in an emergency situation.

U.S. Pat. No. 5,832,488 issued to Eberhardt, discloses a method for storing medical records. The records are entered on a PC and stored on a 3.5 inch diskette. If the file is too large for such a diskette, it is stored in a larger remote data storage computer. The method includes having the patient's regular health care provider input medical information using software installed on the PC, which is then stored either on the diskette, or on the remote computer.

This method has a number of problems. First, the emergency caregiver likely may not have available the type of software necessary to read the medical history saved on the disk. Thus, although the disk can be carried by the patient, it may not, in practice, provide any information to the emergency caregiver.

Second, this system requires the regular health care provider to install new software on its computers whose function it is to create the medical records. This necessitates the hiring of technical support personnel, which is costly. It would also use up valuable memory and computing power on the health care provider's computer, which could possibly be put to better uses.

### SUMMARY OF THE INVENTION

Therefore, what is desired is a method of creating a medical history which does not require the installation of the medical-history-creating software on the computers of the regular health care provider, who is initially in possession of the medical history information. This method will also preferably be useable to create a medical history that is portable by the patient, and thus readily available to an emergency caregiver, without requiring access to a remote source to obtain the medical history. Preferably, the method will create a medical history that will be easily accessible by technology that is widely available, and is thus likely to be available to an emergency caregiver. It will also preferably be a method which creates a complete medical history that is quickly readable by an emergency caregiver.

Thus, according to one aspect of the invention, there is provided a method of creating a conveniently accessible medical history for a patient, the method comprising the steps of:

1) establishing an information-transmission connection with a remote information-input node;

2) receiving medical information through said information-transmission connection from said information-input node;

3) configuring said information into a medical history record; and

4) transmitting said medical history record to a remote record output node;

whereby a conveniently accessible medical history can be created at one location and accessed via a remote record output node.

According to another aspect of the invention, there is provided a method of creating a conveniently accessible medical history for a patient, said method comprising the steps of:

1) establishing an information-transmission connection with a remote record-creating node;

2) transmitting medical information through said information-transmission connection to said record-creating node, said record-creating node being adapted to configure said information into a medical history record, said medical history record being storable on a portable readable storage medium;

Preferably, the method will further comprise the step of receiving said medical history record from said record creating node at said record output node, and storing said medical history record on said portable readable storage medium.

According to another aspect of the invention, there is provided a system for creating a conveniently accessible medical history for a patient, said system comprising:

an information-input node for receiving medical information and transmitting said information through an information-transmission connection;

a record-creating node, remote from said information-input node, for receiving said information through said information-transmission connection, for configuring said information into a medical history record, and for transmitting said medical history record;

a record output node, remote from said record-creating node, for receiving said medical history record from said record-creating node and for permitting access to said medical history record.

According to another aspect of the invention, there is provided a method of creating a conveniently accessible medical history for a patient, the method comprising the steps of:

1) establishing an information transmission connection with a remote information input node;

2) receiving medical information through said information transmission connection from said information input node;

3) configuring said information into a medical history record; and

4) saving, on a portable readable storage medium, a record access certificate for granting remote access to said medical history record; whereby the patient's record may be accessed using the certificate stored on the storage medium.

According to another aspect of the invention, there is provided a method of accessing the medical history record of a patient, the method comprising:

1) reading a record access certificate from a portable readable storage medium;

2) attempting to establish a connection with a remote node holding a primary medical history record;

3) if said connection is established, accessing said medical history record in said remote node, using said record access certificate; and

4) if said connection is not established, reading a backup medical history record from said portable readable storage medium.

### BRIEF DESCRIPTION OF THE DRAWINGS

Reference will now be made by way of example only to drawings of the present invention which illustrate the preferred embodiment of the invention, and in which:

FIG. 1 is a schematic diagram of the system for creating a conveniently accessible medical history according to the present invention;

FIG. 2 is an alternative schematic diagram of the system for creating a conveniently accessible medical history according to the present invention;

FIG. 3 is a schematic diagram of an alternate form of the invention.

### DETAILED DESCRIPTION OF THE PREFERRED EMBODIMENTS

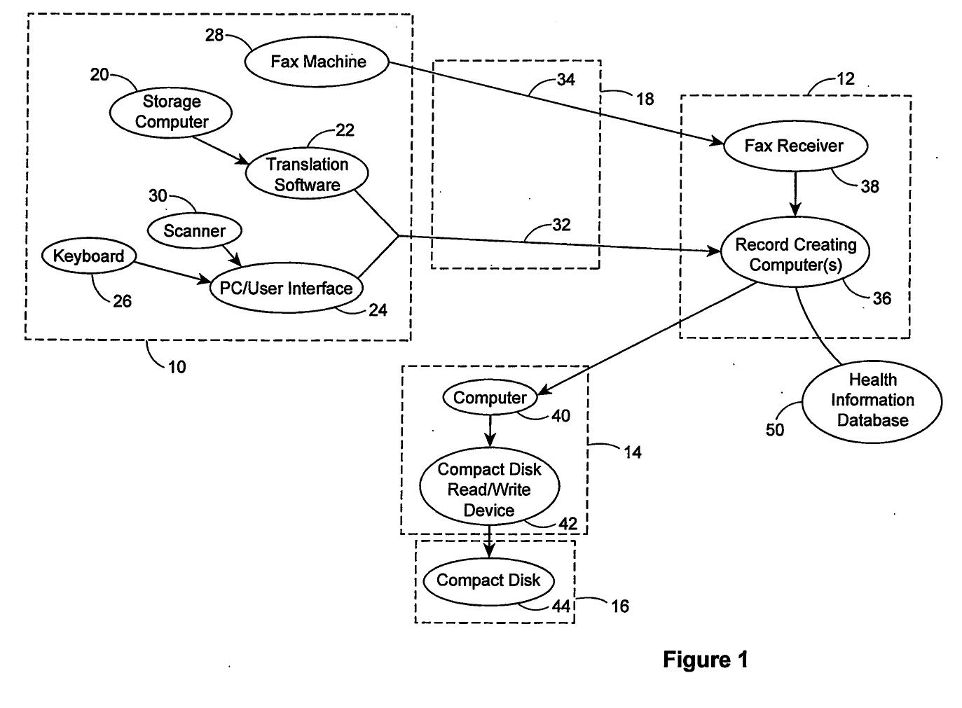

Referring now to FIG. 1, there is shown the preferred embodiment of a system for creating a conveniently-accessible medical history. The system includes an information input node 10, connected to a record creating node 12 by an information transmission connection 18. The record creating node 12 is connected to a record output node 14, which is in turn connected to a portable readable storage medium 16 for storing medical history records.

Generally, the method for creating a conveniently accessible medical history comprises establishing an information-transmission connection 18 between the information input node 10 and the record creating node 12. Medical information is sent from the information input node 10, through the information transmission connection 18, and received by the record creating node 12. The record creating node 12 is adapted to configure the medical information into a medical history record which is storable on a portable readable storage medium. The medical history record is then transmitted by the record creating node 12 and received by the record output node 14. The record output node 14 is configured to store the medical history record on a portable readable storage medium 16. This method is described in greater detail below.

To create a conveniently-accessible medical history, it is necessary to have access to the medical history of the relevant patient. Typically, the medical records of patients will be located at places such as health maintenance organizations (HMOs), doctors' offices, medical clinics or insurance companies (hereinafter referred to generally as “health organization”). Thus, the information input node 10 will typically be located at a health organization. The purpose of the information input node 10 is to contain and send the medical history information which will be used to create the medical history record.

Medical history information can be inputted into the information input node 10 in a variety of ways. Most preferably, the health organization will have patient medical records in electronic form in a storage computer 20, which, for the purposes of this specification, includes any storage media (such as tapes or disks or any other medium) on which the health organization stores raw medical history information in electronic format. This is most likely in cases where the health organization is an HMO. Because HMOs have enormous amounts of medical data from a large number of patients, they are most likely to have invested in storing the data electronically in a storage computer 20. It is most preferable for the patient records to be in electronic form because that obviates the need to input manually the raw medical history information of the patient.

It will be appreciated by those skilled in the art that, even if the raw patient medical data is stored electronically by the health organization, it may not be stored in a format that is compatible with the record creating node 12. In such a case, it would be necessary to create a means for translating the electronic raw medical history information into a format compatible with the record creating node 12. These means would preferably be in the form of translation software 22. The translation software 22 functions to translate the electronically-stored raw medical history information into a data format that can be read by the record creating node 12.

It will be appreciated that the required translation software 22 may need to be customized for each health organization, since each health organization may electronically store its raw medical history information in a different format. The creation of customized translation software 22 is a significant cost for the health organization, as it requires employing people to develop the software. Thus, the use of translation software 22 would likely be cost effective in situations where the health organization is storing a large amount of raw medical history information, and is likely to create a large number of conveniently accessible medical histories using the same translation software 22. In such a case, creating customized translation software 22 would be less costly than inputting raw medical history information by hand.

It will also be appreciated that translation software 22 would not be needed if the raw medical history information is stored by the health organization in a format compatible with the record creating node 12. Preferably, the record creating node 12 will be configured to be compatible with open data format standards such as HL7, ODBC (Open Database Connectivity), XML or HTML. In such a case, if the raw medical history information is stored electronically at the health organization in one of these formats, translation software 22 will not be necessary for translating the raw information from one data format to another.

The raw medical history information will sometimes not be stored in electronic format. Instead, the raw medical history data may be stored on paper. So, for example, the records showing relevant data such as the patient's allergies, past illnesses and current medical conditions might be in type-written or hand-written form on papers contained in the patient's file. In such a case, it might be necessary for this data to be entered manually into the information input node 10 at the health organization.

It will be appreciated that if the raw medical history data is in written form, it will be necessary to manually input the raw data in a manner that makes it compatible with the record creating node 12. In such a case, the information input node 10 would include a PC/user interface 24 and a keyboard 26 connected thereto. The text portion of the raw medical history information could be manually inputted into the PC/user interface 24 through the keyboard 26. The PC/user interface 24 comprises a PC, preferably with a user interface installed thereon. The user interface is preferably simple software, installable on most PCs, which prompts the user to enter the relevant raw medical history information, and stores the entered medical history in a format compatible with the record creating node 12.

It will be appreciated that, in the case where textual medical history information is manually inputted, other configurations besides the PC/user interface 24 might be used to manually input the information. For example, a user interface might be used which is stored at the record creating node 12, and the manual inputting could be done by establishing the information transmission connection 18 and using the PC as a terminal. What is important is that, in the case where textual medical history information is to be inputted manually, the information input node 10 and the record creating node 12 be configured to allow for such manual inputting.

Other relevant patient information includes the results of tests, such as electrocardiograms or various blood work tests. These types of test results are typically shown in graph or chart form, rather than in textual or written form. To be of maximum use to a subsequent caregiver, the entire image is preferably provided. Thus, for this type of medical history information, it is useful to ensure that the entire image (for example, of an electrocardiogram) is available in the medical history record being created.

If the images are stored electronically, they will preferably be dealt with as generally described above with respect to raw medical history data which is stored by the health organization in electronic form. If the images are not in suitable electronic form, the images will need to be inputted and transmitted to the record creating node 12 in order to be included in the medical history record. In such a case, the information input node 10 may include either a fax machine 28 or a scanner 30, or both, which devices can be used to translate medical images into a digital format which allows the record creating node 12 to place these images into the medical history record. As shown in FIG. 1, if a scanner 30 and keyboard 26 are both used, the scanner 30 is preferably connected to the PC/user interface 24. In this way, the user interface can also be used to receive images digitized by the scanner 30.

The information transmission connection 18 links the information input node 10 and the record creating node 12. Medical history information for use in creating a conveniently-accessible medical history, which information may include information entered by hand into the information input node 10, information stored in electronic format, and images, is transmitted via the information transmission connection 18 to the record creating node 12.

Preferably, the information transmission connection 18 comprises an internet connection 32. In other words, the medical information (though not necessarily all of the information) travelling from the information input node 10 to the record creating node 12 will travel via the internet connection 32. This use of the internet is preferred for a number of reasons. First, use of the internet for creating the information transmission connection 18 and transmitting information from the information input node 10 to the record creating node 12 allows for maximum flexibility in the location of the record creating node 12. Given that internet connections 32 are so easily and widely available, the record creating node 12 can be located virtually anywhere. Second, an internet connection is significantly less expensive than, say, a dedicated phone line or a dial-up connection to the record creating node 12.

It will be appreciated that making the information transmission connection 18 a dedicated phone line, or even a dial-up connection, would be more reliable than using the internet connection 32. In other words, an internet connection is likely to be down or slow more often than a dedicated phone line, where the reliability of the connection is extremely high. However, it is believed that reliability of connection is less important than the benefits arising from the use of the internet. The reason for this is that, when the medical history record is being created, there is no emergency situation. Rather, the creation of the record is an administrative task undertaken by the health organization. The task is not undertaken in response to an emergency, and thus there is likely to be no harm to a patient resulting from a slower internet connection, or resulting from the need to wait several minutes before re-attempting the connection. Therefore, the flexibility associated with using the internet makes it preferable to do so.

Nevertheless, it will be appreciated that the information transmission connection 18 may nevertheless comprise a dedicated phone line, dial-up connection, or any other connection which allows for medical history information to be transmitted from the information input node 10 to the record creating node 12.

As discussed above, the information input node 10 may comprise a fax machine 28. If this is the case, then the information transmission connection 18 may also comprise a fax connection 34, between the fax machine 28 and the record creating node 12. If present, the fax connection 34 would most preferably be in parallel to the internet connection 32, in which case the information transmission connection 18 would comprise the internet connection 32 and the fax connection 34 in parallel. Alternatively, the fax connection 34 could also be over the internet. It will be appreciated that, if the fax machine 28 is used, the fax connection 34 can be any connection that links the fax machine 28 to the record creating node 12 so as to allow images to be transmitted along the fax connection 34.

The record creating node 12 is preferably remote from the information input node 10. In this context, “remote” includes the record creating node 12 being in a different location from the information input node 10, or the record creating node 12 being associated with a separate application service provider (ASP), or both. Thus, most preferably, while the information input node 10 is preferably situated on the premises of the health organization, or otherwise under the direct or indirect control of the health organization, the record creating node 12 is preferably “remote” in the sense that it is associated with a separate application service provider (ASP) whose function it is to receive medical history information from the health organization and convert it into a medical history record.

This has the benefit of allowing health organizations to stay out of the business of providing the software, computing power and support personnel necessary to create medical history records. By this model, health organizations would simply be able to use the resources of the ASP's record creating node 12, rather than having to provide their own resources to provide this service. For this reason, the ASP model is more efficient.

Because the same ASP can provide the same record creating services for use by a variety of different health organizations, the ASP will have the advantage of high volume. As a result, it will have the advantage of a relatively low cost for creating each medical history record. By contrast, not having the advantage of high volume, it would cost an individual health organization more to create each medical history record if it created the records on its own without using the ASP. Thus, the health organizations for whom each medical history record is created will have the option of passing the cost (i.e. the price charged to them by the ASP) on to their patients.

Various modes of pricing are available to the ASP. For example, a health organization that frequently uses the ASP could be offered a bulk subscription rate for the record creating node's services. Alternatively, the ASP could charge the health organization a single low price per record created.

Such a system of a flat fee per record created would allow the health organization to easily make a specified level of profit per record created. So, for example, if the ASP charges the health organization five dollars ($5) per record created, the ASP can simply charge the patient ten dollars ($10) per record created, thus turning the record creation into a profit centre for the health organization, in addition to allowing the health organization to pass on the cost of creating the medical history records.

This can be accomplished without complex information or accounting requirements, since all that is required is to multiply the ASP's price charged to the health organization by a specified multiplier to determine what the health organization's price to the customer or patient will be. Also, notably, the ASP will be able to establish this profit centre without investing in the computing resources necessary to create medical history records, the software necessary to do so, and the support personnel that would be necessary to operate such software and hardware. Thus, the ASP model is preferable because, for the reasons just stated, it provides an incentive to health organizations to use the ASP rather than developing their own in-house record-creating capability.

Once the information is transmitted from the information input node 10 to the record creating node 12 through the information transmission connection 18, the record creating node 12 configures the information into a medical history record which is storable on a portable readable storage medium 16. Once the medical history record is stored on the portable readable storage medium 16, a conveniently accessible medical history has been created, because the medical history can be carried by the patient and read by a caregiver when the need arises.

It will be appreciated that the record creating node 12 will preferably comprise one or more record creating computers 36 having record creating software which is configured to take the medical history information received from the information input node 10 and create a medical history record. The record creating computers will preferably be connected to the internet so as to allow for the internet connection 32.

The record creating node 12 will also preferably comprise a fax receiver 38 for receiving faxes from the fax machine 28, if necessary. The fax receiver 38 is connected to the record creating computers 36, and the record creating software is adapted to take faxes received at the fax receiver 38 and incorporate the images in those faxes into the medical history record.

Preferably, the record creating node's record creating software is adapted to configure the medical history information that it receives into a medical history record that is as comprehensive as possible. The more comprehensive the record, the more likely it is to provide effective assistance to a subsequent caregiver attending to the patient. Thus, the medical history record created at the record creating node 12 will most preferably include at least: the patients name, address and phone numbers; allergies; allergic reactions; active medications; dosages; frequency of taking medications; start date of the medications; any “alternative” medicines being taken; any active conditions that have been diagnosed; the date that the conditions began; any diagnoses of past conditions; any past surgeries; emergency contact names; the particulars of how and where to reach those contacts; the name and particulars of the patent's primary care physician; a full personal profile of the patient, including a full physical description, religion, employment, martial status, insurance particulars, whether a smoker, family medical history, blood type, age and sex; the results of tests, including haematology and biochemistry test results, diagnostic image reports (e.g. CT scans, nuclear medicine, x-ray, ultrasound and MRI), electrocardiograms, echocardiograms, stress tests, coronary angiographies, catheterization reports and holter monitoring reports; the patient's immunization history, transplant history, and a full record of any implantable devices (e.g. pacemaker) that have been implanted in the patient; and a photograph of the patient, which adds a further check for insuring that the medical history record really belongs to the person carrying it. The record will preferably include the particulars of the patient's primary care physician, including his name, address and contact information.

In the preferred embodiment, the record creating computer 36 is coupled to a health information database (HID) 50. The HID 50 will contain information on various different medical conditions, including information related to symptoms, treatment, nutrition, surgery and management of the condition.

In creating the medical history record, the record creating computer 36 will preferably read the medical history information to ascertain what conditions the patient suffers from, is predisposed to, or is at risk from. The computer 36 will then extract from the HID 50 information relating to these medical conditions and include such information in the medical history record.

In the preferred embodiment, the medical condition information will be linked to the statement of the condition itself within the medical history record. So, for example, if the patient has asthma, the word “asthma” would appear in the medical history record. The patient would be able to click on the word “asthma” and be linked to medical condition information on asthma. The same would be true for other conditions that the patient has, is at risk from, or is predisposed to.

After the medical history record is created within the record creating node 12, it is transmitted to a record output node 14. The record output node 14 is configured to store the medical history record, which has just been created in the record creating node 12, on the portable readable storage medium 16. It will be appreciated that the medical history record is preferably transmitted via the internet, which, as stated above, provides an inexpensive and flexible way of transmitting the medical history record to the record output node 14.

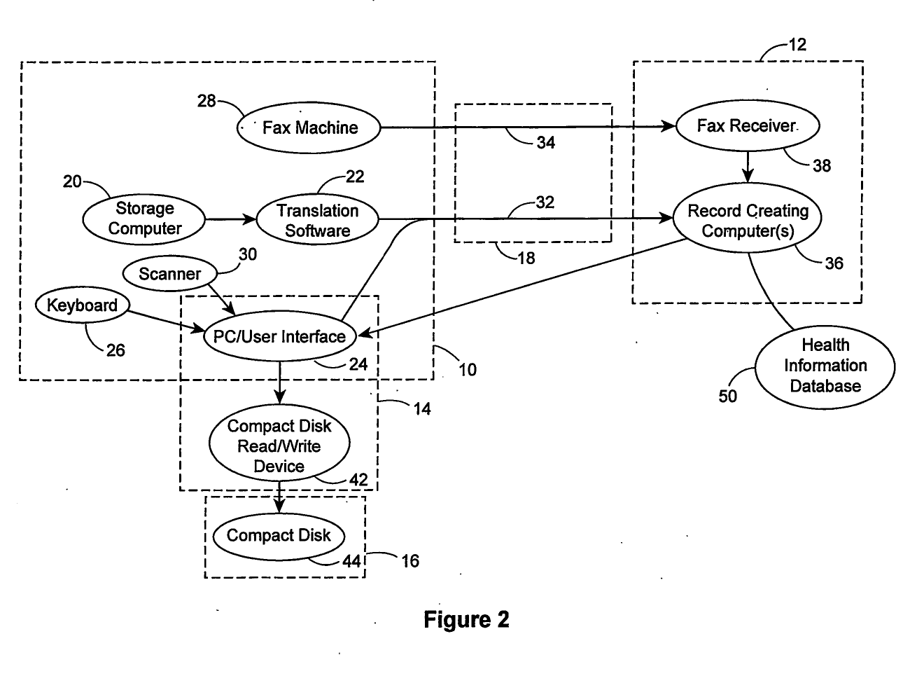

It will be appreciated that the record output node 14 and the information input node 10 may include a single computer at the premises of the health organization. This configuration is shown in FIG. 2, with like elements being designated by the same reference numerals as in FIG. 1. Conceptually, the record output node 14 includes, at least, the software and hardware which is needed to store the medical history record on the portable readable storage medium 16. Thus, for example, the record output node 14 may include the PC in the PC/user interface 24. In this case, the PC/user interface would include the software and hardware which is needed to store the medical history record on the portable readable storage medium 16, such as, preferably, a compact disk read/write device.

Alternatively, as shown in FIG. 1, the record output node 14 may comprise an output computer 40, which is separate from the PC/user interface 24, connected to a compact disk read/write device 42.

Thus, it will be appreciated that, when reference is made to a record output node 14 and to an information input node 10, these two elements may be associated with the same computers and hardware (such as the PC/user interface), or with separate computers and hardware (as shown in FIG. 1). What is important is that the functions of information input node 10 and of the record creating node 14 are performed in creating the conveniently accessible medical history.

Preferably, the portable readable storage medium 16 is a compact disk 44, and most preferably, the compact disk 44 will be approximately the size of a conventional credit card, so as to be easily carried in a wallet. The use of a compact disk 44 is preferred because most personal computers sold today include compact disk readers. Thus, a hospital seeking to make use of a medical history record stored on a compact disk will be able to do so, without being required to engage in costly technology upgrades. Rather, it is likely that they will be able simply to use the computers that are already present in the hospital. This same benefit will accrue to emergency medical workers in the field, such as paramedics, who would be able to read the medical history record from a compact disk with an ordinary laptop computer. Thus, even in outlying or remote areas, paramedics would have a means of reading the medical history record.

It will be appreciated by those skilled in the art that compact disks are also preferred because they are capable of storing a great deal of data (currently about 650 MB for a regular compact disk and 30-50 MB for a credit card-sized disk), as compared to, say, a three-and-one-half inch (3½″) diskette. It will also be appreciated that compact disks can be read more quickly by a computer than many other electromagnetic storage media, such as tapes or diskettes. Finally, compact disks are preferred because they are available in sizes which are approximately similar to the size of a conventional credit card. This allows the compact disk to be easily carried in a wallet, purse or pocket of the patient, so as to be easily available to emergency medical workers who require access to a medical history record.

Nevertheless, it will be appreciated that the portable readable storage medium 16 can be any storage medium which can be carried by a patient and accessed by a subsequent caregiver treating the patient, such that the medical history record stored thereon is conveniently accessible. What is important is that the caregiver be able to conveniently access the medical history record stored thereon.

Because the medical history record is portable, it provides an advantage with respect to security. In other systems, where the medical history record might be accessed from a remote location, confidentiality would require the use of some kind of password. However, in an emergency situation, the patient might not be able to provide that password. According to the present invention, the medical history record is preferably carried on the person of the patient. Thus, there little risk that the medical history record will be accessed without the permission and knowledge of the patient, unless the patient is unconscious, ill or injured, in which case the emergency medical workers will simply access the record being carried by the patient. That is the purpose of having the patient carry the medical history record. Since the medical record is being carried on the person of the patient, it is not available to be accessed for inappropriate reasons (absent loss or theft), and thus no password is generally required.

The medical history record created in the record creating node 12 is preferably represented in either HTML or XML. A record in HTML would be readable by any ordinary internet browser software, and newer internet browsers will be able to utilize XML. Most personal computers are sold with internet browser software, such as Microsoft's Internet Explorer, or Netscape's internet browser. Thus, if the medical history record is represented in HTML or XML, it is likely that any computers used by emergency medical personnel will be able to read the medical history record, without requiring any additional software, thus saving expense and facilitating use of the medical history record.

It will be appreciated that, if XML is used, it may be possible for a hospital to upload the medical history record from the compact disk directly into the hospital's electronic medical record. Specifically, this would be possible if the hospital's electronic medical record is itself represented in XML. Thus, a patient arriving at the emergency room of the hospital can have his medical history record directly uploaded into the hospital computer, thus reducing the amount of paperwork and data entry needed at the hospital.

It will also be appreciated, however, that the medical history record need not necessarily be represented in HTML or XML. Rather the medical history record may be in any format that allows it to be read by a subsequent caregiver.

Thus, in the preferred embodiment where the portable readable storage medium 16 is a compact disk, the record output node 14 will preferably include a computer 40 which is connected to a compact disk read-write device 42. It is in this way that the record output node 14 stores the medical history record on the portable readable storage medium 16, which is preferably a compact disk.

As one of the purposes of the medical history record is to allow for emergency medical personnel to have access to the medical history of a patient, it is important that the medical history record be accurate. Thus, it is preferable that provision be made for the medical history record to be inspected after being created in the record creating node 12, but before being stored by the record output node 14 on the portable readable storage medium 16. Thus, preferably, the record output node 14, and more specifically, the output computer 40, is configured so as to allow the medical history record to be displayed and inspected, preferably by a medical professional, prior to being saved on the portable readable storage medium 16.

The use of the internet in the preferred embodiment to transmit medical history information from the information input node 10 to the record creating node 12, and from the record creating node 12 to the record output node 14 raises confidentiality concerns. The internet is generally not secure, and information transmissions over the internet can be intercepted. Thus, it is preferable that information transmitted from the information input node 10 and from the record creating node 12 be encrypted. Most preferably, the encryption technology used will be PKI encryption.

Also, the encryption will preferably be 128-bit encryption. Presently, technology for conducting secure 128-bit encrypted communication over the Internet is easily available commercially, but still provides a level of encryption which is difficult to defeat.

Thus, the record creating node 12 is preferably configured to encrypt the medical history record prior to transmitting it to the record output node 14, and the record output node 14 is adapted to decrypt the medical history record. Also, the information input node 10 preferably is configured to encrypt medical history information being sent over the information transmission connection 18, and the record creating node 12 is preferably configured to decrypt information coming over the information transmission connection 18.

In the preferred embodiment, the raw medical history information is encrypted at the information input node 10 using the ASP's public key and decrypted by the ASP using its private key. The raw medical history information is then configured into a medical history record at the record creating node 12. Then, the medical history record is encrypted using the health organization's public key. One copy is transmitted to the record output node 14 and decrypted by the health organization's private key. Another is retained in encrypted form on the ASP server. This allows the health organization to update the medical history record at any time by simply sending the new raw medical history information to be incorporated. However, because the retained copy is encrypted, the ASP cannot access the data without first having it decrypted by the health organization.

In some circumstances, it may be desirable to provide primary access to the medical history record at the database located at the ASP, rather than via the portable readable storage medium 16. This may be so because, in certain situations, the database copy of the medical history record may be more up-to-date than the one on the portable readable storage medium. For example, doctors sometimes send patients to laboratories for tests. The test results are sent to the doctor at a later time. When the lab results become available, the patient and his portable readable storage medium 16 are not present. Thus, the copy of the record on the medium 16 cannot be updated. However, the doctor can still, conveniently, update the patient's record on the database in the patient's absence, and may do so, resulting in the database containing a more up-to-date medical record than the medium 16.

In this alternative embodiment, the medical information is received at the ASP and configured into a medical history record, as described above. Then, a record access certificate, preferably in the form of the patient's PKI private key, is transmitted to the remote record output node 14 and stored on the portable readable storage medium 16. A copy of the medical history record may optionally be transmitted to the record output node 14 and a secondary copy thereof saved on the portable readable storage medium 16.

In this alternative embodiment, when the patient attends at a hospital emergency room, or a consulting physician (e.g. a specialist) other than his primary care physician, and access to the patient's medical history record is desired, the record can be accessed at a database server 37 associated with (or comprising) the record creating computer 36 of the ASP which holds an up-to-date, primary, copy of the medical history record. For security purposes, the database server's copy of the record is encrypted using the patients public key. Thus, the record access certificate saved on the portable readable storage medium can be used to grant remote access to the record saved on the database server by, inter alia, permitting decryption of the record;

Thus, to obtain access to the record saved on the database server, the record access certificate is read from the portable readable storage medium 16. A connection to the database server 37 of the remote record creating node 12 of the ASP is attempted. If the connection is established, the record on the database server is accessed using the record access certificate. If the connection is not established, then the copy of the medical history record saved on the portable readable storage medium 16 is accessed instead.

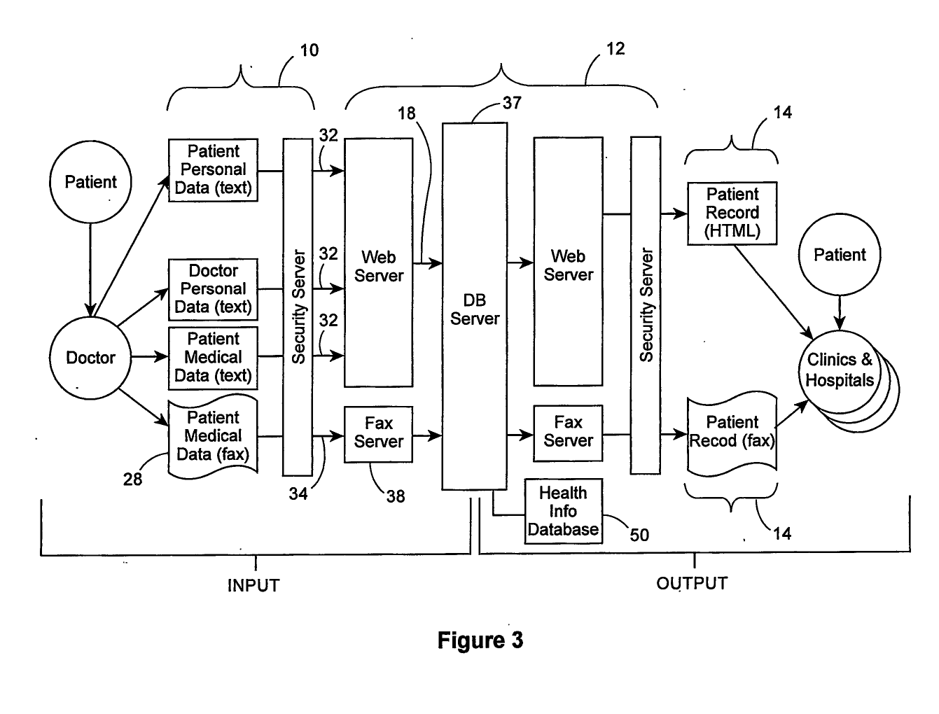

Another alternative embodiment is shown in FIG. 3. This embodiment may be desirable for use in regulatory environments in which centralized storage of medical history records is mandated or encouraged. In this embodiment, no portable readable storage medium 16 is used. Rather, the medical history record is stored in the record creating node 12 after it is created as described above. The record creating node 14 is adapted to configure the medical information received into a medical history record and to transmit the medical history record to a remote record output node 14.

When a hospital, specialist, or other caregiver requires access to the medical history record, it establishes a connection with the server 36 and accesses the medical history record remotely. The medical history record is transmitted to and received by a remote record output node 14, which in this alternate embodiment is typically a computer or computer network located at the premises of the hospital, specialist or other caregiver that is accessing the medical history record. Thus, while in the embodiments described above the record output node 14 permits access to the medical history record via the portable readable storage medium 16 (either by saving the record thereon, or by providing a record access certificate), in this alternate embodiment, access to the record is provided via the record output node, either on a screen, on a printer, by fax or by another means.

It will be appreciated that, in this embodiment, the patient does not exercise control over access to his medical history record by virtue of his physical custody of a portable readable storage medium 16. Therefore, for the purposes of privacy and security, the patient will preferably be permitted to select a set of permitted record accessors, in the form of a list of care providers that may access the patient's medical history record. This list of permitted record accessors will be transmitted from the information input node 10 to the record creating node 12. Then the medical history record, after it is created, is encrypted using the public keys of at least one of, and preferably each of, the permitted record accessors. Then, when a particular caregiver seeks access to the medical history record, it can decrypt the medical history record using its private key, if it is contained in the set of permitted record accessors. If not, it cannot decrypt the record and cannot have access to it. In this way, the patients privacy, and the security of his medical history record, are protected.

It will also be appreciated that a patient may wish to give certain caregivers access to part but not all of his medical history record. This enhances the privacy protection for the patient. Thus, for example, the patient may grant his eye-doctor access to the medical history record by specifying the eye-doctor as one of the permitted record accessors. However, the patient's medical history record may include previous psychiatric conditions which the patient would like to keep as private as possible, and not disclose to the eye-doctor. By contrast, the patient's family doctor and psychiatrist would probably require access to this information, so the patient would want them to have access to the psychiatric history portion of the medical history record.

Thus, when the record is being created, the patient will preferably (in this alternative embodiment) specify, for each permitted record accessor, at least one portion of the medical history record to which access is permitted for the corresponding permitted record accessor. This “portion” may be the entire medical history record. The patient will also preferably be able to update this specification after the record has been created. In this way, the patient can create a system of role-based access for his caregivers, wherein each caregiver has access to the portions of the medical history record that he needs to know in order to give effective care, or that it is desirable for him to know.

Preferably, this selection is implemented as follows. When the patient selects the set of permitted record accessors and the portions of the medical history record accessible to each permitted record accessor, each portion of the medical history record is encrypted using only the public key of each permitted record accessor permitted to access that portion of the medical history record. As a result, each permitted record accessor can only decrypt (using his private key) the portion or portions of the medical history record to which the patient has permitted him access. For example, if the eye-doctor has been selected by the patient to have access to the “allergies” portion of the medical history record but not the psychiatric history portion, then the “allergies” portion will be encrypted using the eye-doctor's public key, but the psychiatric history will not. Since the medical history record is encrypted on the database, when the eye-doctor decrypts the medical history record using his private key, he will successfully decrypt only those portions of the record which he is permitted to access (including the “allergies” section), but not those portions (including the psychiatric history) to which access is not permitted.

While the foregoing embodiments of the present invention have been set forth in detail for the purposes of making complete disclosure of the invention, it will be apparent to those skilled in the art that various modifications can be made to the invention without departing from the scope of the invention as defined in the attached claims. Some of these variations are discussed above and others will be apparent to those skilled in the art. For example, the medical history record may be represented in any format that can be read by a subsequent caregiver such that the caregiver has access to the medical history record. Also, the record output node may be configured to cause at least a part of the medical history record to be printed on a printer or to be accessible via a wireless communication device. Also, the portable readable storage medium may be anything that can store, and allow the reading of, at least a record access certificate and preferably the medical history record. What is important is that the medical history be conveniently accessible so as to improve the care given to the patient.

1. A method of creating a conveniently accessible medical history for a patient, said method comprising the steps of:
1) establishing an information-transmission connection with a remote information-input node;
2) receiving medical information through said information-transmission connection from said information-input node;
3) configuring said information into a medical history record; and
4) transmitting said medical history record to a remote record output node which record output node is configured to receive said medical history record; whereby a conveniently accessible medical history can be created at one location and accessed via a remote record output node.

2-22. (canceled)

23. A method of creating a conveniently accessible medical history for a patient, said method comprising the steps of:
1) establishing an information-transmission connection with a remote record-creating node;
2) transmitting medical information through said information-transmission connection to said record-creating node, said record-creating node being adapted to configure said information into a medical history record and to transmit said medical history record to a remote record output node.

24. The method of claim 23, further comprising the step of receiving said medical history record from said record-creating node at said record output node.

25. The method of claim 24, wherein said step 2 comprises the step of transmitting to said record creating node, said record creating node being adapted to configure said information into a medical history record that is storable on a portable readable storage medium, the method further comprising the step of storing said medical history record on said portable readable storage medium.

26-28. (canceled)

29. The method of claim 25, further comprising the step of inspecting said medical history record prior to the storing step.

30. The method of claim 25 the storing step comprising storing the medical history record on a compact disk.

31. The method of claim 25, the storing step comprising storing the medical history record on a compact disk which is approximately the size of a conventional credit card.

32-43. (canceled)

44. A system for creating a conveniently accessible medical history for a patient, said system comprising: an information-input node for receiving medical information and transmitting said information through an information-transmission connection; a record-creating node, remote from said information-input node, for receiving said information through said information-transmission connection, for configuring said information into a medical history record, and for transmitting said medical history record; a record output node, remote from said record-creating node, for receiving said medical history record from said record-creating node and for permitting access to said medical history record.

45. The system of claim of claim 44, the record output node being configured to store said medical history record on a portable readable storage medium.

46-48. (canceled)

49. The system of claim 45, said record output node being configured to permit inspection of said medical history record by a medical practitioner prior to said medical history record being stored on said portable readable storage medium.

50. The system of claim 45, wherein said portable readable storage medium is a compact disk.

51. (canceled)

52. The system of claim 44, said information-input node comprising a computer.

53. The system of claim 52, said information input node further comprising a fax machine, said information-transmission connection further comprising a connection between said fax machine and said record-creating node.

54-57. (canceled)

58. A method of creating a conveniently accessible medical history for a patient, the method comprising the steps of:
1) establishing an information transmission connection with a remote information input node;
2) receiving medical information through said information transmission connection from said information input node;
3) configuring said information into a medical history record; and
4) saving, on a portable readable storage medium, a record access certificate for granting remote access to said medical history record; whereby the patient's record may be accessed using the certificate stored on the storage medium.

59. The method of claim 58, further comprising the step of saving said medical history record on said portable readable storage medium.

60. The method of claim 59, further comprising prior to the step of saving said medical history record, the step of transmitting said medical history record to a remote record output node.

61. The method of claim 58, further comprising, prior to the step of saving the record access certificate, the step of transmitting the record access certificate to the remote record output node.

62. The method of claim 58, said step 4 comprising the step of saving, on said portable readable storage medium, a PKI certificate of said patient, for granting access to said medical history record.

63. The method of claim 58, wherein said configuring step further includes including in said medical history record medical condition information relating to a medical condition of the patient, the medical condition information including information on symptoms and treatment of the medical condition.

64. A method of accessing the medical history record of a patient, the method comprising:
1) reading a record access certificate from a portable readable storage medium;
2) attempting to establish a connection with a remote node holding a primary medical history record;
3) if said connection is established, accessing said medical history record in said remote node, using said record access certificate; and
4) if said connection is not established, reading a backup medical history record from said portable readable storage medium.
